(19) United States
(12) Patent Application Publication
(10) Pub. No.: US 2003/0211503 A1
(43) Pub. Date: Nov. 13, 2003

(54) Materials and methods for the diagnosis of pediatric tumors

(76) Inventor: David F. Carpentieri, Paradise Valley, AZ (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/262,391

(22) Filed: Oct. 1, 2002

Related U.S. Application Data
(60) Provisional application No. 60/326,303, filed on Oct. 1, 2001.
(60) Provisional application No. 60/401,974, filed on Aug. 8, 2002.

(51) Int. Cl. C12Q 1/68; G01N 33/574

(52) U.S. Cl. 435/6; 435/7.23

(57) ABSTRACT

The present invention is directed to methods wherein the cytolocalization of WT1 protein can be used as a tool in the differential diagnosis of soft tissue tumors. Specifically, the invention is directed to the novel finding that elevated levels of WT1 protein in the cytoplasm of cells derived from a soft tissue tumor sample provide a positive diagnostic indicator for rhabdomyosarcoma (RMS).

This application claims priority to U.S. provisional applications 60/326,303 and 60/401,974 filed Oct. 1, 2002 and Aug. 8, 2002 respectively. The entire disclosure of these applications is incorporated by reference herein.

### FIELD OF THE INVENTION

This invention relates to the fields of molecular biology, pediatric oncology, and pathology. More specifically, the invention provides materials and methods for facilitating diagnosis of pediatric tumors. Methods are provided for detecting WT1 protein levels and cytolocalization, which are useful in the diagnosis of rhabdomyosarcoma. Also provided are methods to detect WT1 RNA transcript levels in patient sera, levels of which are indicative of the therapeutic efficacy of a cancer treatment. Kits are also provided to facilitate the practice of methods of the invention.

### BACKGROUND OF THE INVENTION

Several publications and patent documents are referenced in this application by numerals in parentheses or by patent number in order to more fully describe the state of the art to which this invention pertains. The disclosure of each of these publications and documents is incorporated by reference herein.

The WT1 gene (13) encodes a protein with four zinc fingers of the Kruppel-type in the C-terminal region that recognizes a guanidine-cytidine (GC)-rich “EGR1” consensus sequence (17) required in tissue differentiation and proliferation (17, 35, 46). The N-terminal half contains a large proline/glutamine-rich domain important for inhibition of transcriptional activation (8, 44). There are at least eight protein isoforms ranging between 52-62 kD in mammals produced by a combination of alternative splicing and RNA editing (18).

The WT1 proteins are normally expressed in the nuclei of glomerular podocytes and mesothelial cells. They are also present in stem cells bearing the CD34+ phenotype (3). The role of WT1 in normal human development extends to a diversity of mammalian mesodermal tissues (2), including the body-wall musculature at 13.5 days post-conception. Embryologic studies of wt1-null mice reveal a failure to develop kidney and gonads (23). Mutations and splicing disruptions of WT1 have been described in Denys-Drash (7, 27, 40, 55), WAGR (38) and Frasier (22, 40) syndromes.

Rhabdomyosarcoma (RMS), the most common soft tissue sarcoma in children younger than the age of 15 years, is included within the broad classification of SRBCT. Histologically, RMS can be subdivided into two major subtypes; embryonal (E-RMS), which is more common, and alveolar (A-RMS) rhabdomyosarcoma. Immunohistochemistry has been used extensively to distinguish RMS from similarly presenting tumors. Myogenin and MyoD1, myogenic transcriptional regulatory proteins expressed early in skeletal muscle differentiation, are considered to be the most sensitive and specific markers of RMS. Myogenin and MyoD1 have been found to be more specific than desmin and muscle-specific actin and more sensitive than myoglobin in the diagnosis of RMS.

The expression patterns of myogenin and MyoD1 have also been examined in rhabdomyosarcoma subtypes and spindle cell tumors and are considered to be of utility in the differential diagnosis of RMS. A recent survey wherein formalin-fixed, paraffin-embedded archival tissue from RMS, non-RMS, and benign skeletal muscle samples were stained for myogenin and MyoD1 with standard immunohistochemical techniques (57) revealed that all RMS tumors expressed myogenin. Alveolar RMS (A-RMS) showed strong nuclear myogenin staining, especially in tumor cells lining fibrous septae and perivascular regions. E-RMS tumors were, however, more variable in myogenin staining pattern and intensity. No cases of nodular fasciitis, malignant fibrous histiocytoma, malignant peripheral nerve sheath tumor, inflammatory myofibroblastic tumor, myofibrosarcoma, leiomyoma, leiomyosarcoma, or alveolar soft part sarcoma stained for myogenin. Focal nuclear reactivity, however, was seen in desmoid, infantile myofibromatosis, synovial sarcoma, and infantile fibrosarcoma. Moreover, non-neoplastic skeletal muscle fiber nuclei stained positively for myogenin in both tumor-associated samples (25 of 40) and benign skeletal muscle samples (5 of 11). In view of the variability in myogenin staining for the most common form of RMS, E-RMS, and the presence of myogenin staining in rare non-RMS tumors and normal skeletal tissue, it is clear that methods for detecting myogenin fail to provide a clinician with the means to definitively diagnose RMS.

Methods directed toward detection of MyoD1, another preferred and specific marker of RMS, also fail to provide a definitive diagnostic tool indicating RMS. For example, all RMS tumors tested by Chen et al. were immunoreactive for MyoD1. However, cytoplasmic and nonspecific background staining and reactivity of nonmyoid tissues were found to hinder its practical utility in paraffin-embedded samples (57).

The above findings underscore the drawbacks of methods based solely on myogenin or MyoD1 detection for the diagnosis of RMS. Clearly, a need exists for improved methods and more reliable markers for definitively diagnosing RMS.

### SUMMARY OF THE INVENTION

The present invention provides methods for diagnostic evaluation and genetic screening of patients at risk for, or currently suffering from cancer. Specifically, the invention provides methods with which to differentiate rhabdomyosarcomas from other soft tissue tumors and thereby provides a clinician with more definitive means with which to diagnose a patient with rhabdomysosarcoma. The ability to distinguish rhabdomysosarcoma from other soft tissue tumors, particularly Wilms' tumors, is of considerable utility in determining an appropriate course of therapeutic intervention for a patient. Differential diagnosis of rhabdomyosarcoma and Wilms' tumor is critical for prognostic outcome, as the treatment regimes recommended for treatment of patients diagnosed with these different tumors are fundamentally dissimilar.

The present inventor has discovered that the presence of elevated cytoplasmic staining of the Wilms' tumor protein (WT1) in a soft tissue tumor provides a positive diagnostic indicator of rhabomyosarcoma. Accordingly, methods are provided for localizing WT1 in tumor samples from patients suspected of having RMS. Also provided are methods for the detection of WT1 messenger RNA (mRNA) in peripheral blood and sera derived from patients undergoing chemotherapeutic, radiation, and/or combination therapy. The present inventor has also discovered that monitoring the levels of WT1 message in peripheral blood and/or sera obtained from RMS patients provides an accurate gauge with which to evaluate the efficacy of such therapeutic intervention. A decrease in circulating WT1 mRNA levels is indicative of a reduction in the cancer cell load, whereas maintenance of high WT1 mRNA provides indication that the treatment method should be altered to optimize targeting of diseased cells.

In another aspect of the invention, periodic evaluation of circulating WT1 mRNA levels in patients in remission may also provide means to diagnose residual disease and/or disease relapse. Moreover, periodic evaluation of circulating WT1 levels in relatives (e.g., siblings) of rhabdomyosarcoma patients may be of utility in the early detection of disease onset. Periodic evaluation of siblings is particularly useful because epidemiological studies of childhood rhabdomysarcoma and other soft tissue sarcomas of childhood have revealed a familial disposition (58, 59, 60).

As disclosed herein, the detection of elevated levels of cytoplasmic WT1 in tumor cells by immunohistochemical methods may be used for the differential evaluation of patients presenting with soft tissue tumors. Soft tissue tumors include, but are not limited to, Wilms' tumor, angiomyoma, melanoma, myeloma, neuroblastoma, glioma, schwannoma, leiomyoma, and sarcoma (i.e., rhabdomyosarcoma, chondrosarcoma, and myofibrosarcoma). The methods of the invention are of particular utility in the differential diagnosis of rhabdomyosarcoma and Wilms' tumor. When combined with staining protocols to detect muscle specific proteins (e.g., myogenin, MyoD1, desmin, muscle specific actin, or myoglobin), elevated WT1 cytoplasmic staining provides a definitive positive diagnostic indicator of rhabdomyosarcoma.

In a preferred embodiment of the invention, immunohistochemistry methods are provided for detecting high levels of WT1 protein localized to the cytoplasm of cells in biological samples. Other embodiments of the invention include methods such as polymerase chain reaction (PCR) using WT1 nucleic acid molecules as primers to detect WT1 message levels in peripheral blood and sera.

In yet another aspect of the present invention, kits are provided for practicing the methods set forth above. An exemplary kit for screening tumor samples for WT1 protein expression includes reagents for immunological detection of WT1. For example, such kits may include immobilized WT1 protein and antibodies immunologically specific for WT1. Such kits may be used for immunohistochemical assessment of biopsy specimens for detection, quantification, and/or localization of WT1 in biological specimens, particularly specimens of soft tumor tissue biopsies. Another exemplary kit employs PCR methodology. Kits of this type include reagents for performing PCR, such as suitable primers for PCR amplification of target WT1 sequences, polymerase enzyme, and suitable buffers. Exemplary primers include those having the sequence of SEQ ID NO: 1, SEQ ID NO: 2, SEQ ID NO: 3 or SEQ ID NO: 4. A kit in accordance with the invention may also contain vials, a target WT1 sequence as a positive control and a protocol sheet.

### BRIEF DESCRIPTION OF THE DRAWINGS

FIG. 1 depicts the results of immunohistochemistry analyses using WT1 (6F-H2) antibodies. A) Normal kidney positive podocytes; B) 8 weeks fetus intestinal wall with positive mesothelial cells; C) 8 weeks fetus hand negative skeletal muscle staining; D) Wilms' tumor with epithelial and blastemal nuclear positivity; E) Wilms' tumor with muscle differentiation showing strong cytoplasmic reactivity; F) Rhabdomyosarcoma with elevated cytoplasmic expression.

FIG. 2 shows a Western blot of WT-1 in a rhabdomyosarcoma (RMS) tumor specimen. A band of 52 kD that co-migrates with either transfected WT-1 (293/WT-1B) or endogenous WT-1 (293, Wilms' tumor) is detected in the rhabdomysarcoma sample. Equal amounts of protein are loaded as indicated by similar levels of tubulin expression.

FIG. 3 is a photograph of a gel following electrophoretic separation of WT1 specific PCR products generated by RT-PCR of samples derived from a rhabdomyosarcoma (RMS) patient: Peripheral blood (PB @ 1:1 and 1:100 dilution) and RMS tissue (1:50 and 1:100) co-migrate with controls (ALL patient and K562 cell line).

### DETAILED DESCRIPTION OF THE INVENTION

The WT1 gene encodes a transcription factor implicated in normal and neoplastic development. A mouse monoclonal antibody (clone: 6F-H2, Dako) raised against the N-terminal amino acids 1-181 of the human WT1 protein was tested on a variety of pediatric small round blue cell tumors. Microscopic sections from 66 specimens were stained using an antigen retrieval protocol with trypsin. The tumors included 8 peripheral neuroectodermal tumors (PNET/Ewing's), 8 neuroblastomas, 5 desmoplastic small round cell tumors (DSRCT), 10 lymphomas, 24 Wilms' tumors and 11 rhabdomyosarcomas. An 8 weeks old human fetus and a normal kidney were used as controls. A rhabdomyosarcoma case was investigated by Western blot analysis and RT-PCR.

As disclosed herein, elevated cytoplasmic staining was demonstrated in all rhabdomyosarcoma samples examined. Wilms' tumors exhibited a variable nuclear (usually epithelial or blastemal) and/or cytoplasmic (usually stromal) positivity in 92% (22/24) of the cases. Nuclear positivity was recorded in 58% (14/24) of Wilms' tumors, whereas the cytoplasmic pattern was seen in 75% (18/24) of Wilms' tumors. Some DSRCT (3/5), lymphomas (4/10) and neuroblastomas (2/8) displayed a weak (1+ to 2+) nuclear or cytoplasmic staining. All PNET/Ewing's were negative. The presence of WT1 protein in tissue samples was confirmed by Western blot and detection of WT1 RNA by RT-PCR in tissue and blood derived from a patient diagnosed with a rhabdomyosarcoma is described.

An elevated WT1 cytoplasmic expression pattern in a soft tissue tumor, therefore, provides a positive diagnostic indicator of rhabdomyosarcoma, whereas an elevated WT1 nuclear staining pattern is suggestive of Wilms' tumor. Accordingly, these results provide the basis for a rapid diagnostic method for differentiating between these two tumor types in pediatric patients. Thus, in combination with staining to detect myogenin, MyoD1, desmin, muscle specific actin, or myoglobin, elevated WT1 cytoplasmic staining provides a definitive positive diagnostic indicator of rhabdomyosarcoma.

The discovery that an elevated WT1 cytoplasmic expression pattern in a pediatric soft tissue tumor is indicative of rhabdomyosarcoma provides a powerful diagnostic tool for clinicians. Rhabdomyosarcoma is the most common soft tissue sarcoma in children younger than the age of 15 years. Accurate diagnosis of RMS is required to evaluate treatment modalities such as surgical resection, radiotherapy and chemotherapy. The markers that are currently utilized to diagnose RMS are not universally reliable, as underscored by the potential for false positive results that may confound definitive diagnosis. Accordingly, accurate histological classification of RMS tumors has prognostic relevance and should aid in the selection of appropriate therapy.

In one embodiment, the methods of the present invention may be used to advantage in the diagnosis of rhabdomyosarcoma. In another embodiment, the methods of the invention may be of utility in evaluating the efficacy of a therapeutic regime to eradicate a rhabdomyosarcoma and/or detecting minimal residual disease and/or relapse following treatment. In another embodiment, the methods of the invention may be useful for predicting the potential for disease onset in a relative of a patient diagnosed with rhabdomyosarcoma.

The term “isolated nucleic acid” is sometimes used with reference to nucleic acids of the invention. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the genomic DNA of a prokaryote or eukaryote.

When used with reference to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a substantially pure form.

The terms “isolated protein” or “isolated and purified protein” are sometimes used herein to refer to a protein produced by expression of an isolated nucleic acid molecule of the invention. Alternatively, these terms may refer to a protein that has been sufficiently separated from other proteins with which it would naturally be associated, so as to exist in substantially pure form.

The term “substantially pure” refers to a preparation comprising at least 50-60% by weight of the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like).

With respect to nucleic acids and oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). When used in reference to a double stranded nucleic acid, this term is intended to signify that the double stranded nucleic acid has been subjected to denaturing conditions, as is well known to those of skill in the art. In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “oligonucleotides,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

One common formula for calculating the stringency conditions required to achieve hybridization between nucleic acid molecules of a specified sequence homology Sambrook et al. (61):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such sequences would be considered substantially homologous to the nucleic acid sequences of the invention.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

A “cell line” is a clone of a primary cell or cell population that is capable of stable growth in vitro for many generations.

An “immune response” signifies any reaction produced by an antigen, such as a viral antigen, in a host having a functioning immune system. Immune responses may be either humoral in nature, that is, involve production of immunoglobulins or antibodies, or cellular in nature, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems. Such immune responses may be important in protecting the host from disease and may be used prophylactically and therapeutically.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunoglobulin molecule such as those portions known in the art as Fab, Fab′, F(ab′)2, F(v) and Sfv generated recombinantly.

With respect to antibodies, the term “immunologically specific” refers to antibodies that bind to one or more epitopes of a protein (e.g., WT1) or compound of interest, but which do not substantially recognize and bind other molecules in a sample containing a mixed population of antigenic biological molecules.

The phrase “WT1 associated molecule” refers to a WT1 protein, polypeptide or fragment thereof. The phrase also encompasses WT1 encoding nucleic acids or fragments thereof. Such nucleic acids may be DNA, cDNA or RNA.

I. Preparation of Nucleic Acid Molecules, Probes and Primers

Nucleic acid molecules encoding the oligonucleotides of the invention may be prepared by two general methods: (1) synthesis from appropriate nucleotide triphosphates, or (2) isolation from biological sources. Both methods utilize protocols well known in the art. The availability of nucleotide sequence information, such as the DNA sequence encoding WT1 (Genbank No. XM 034418), enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be used directly or purified according to methods known in the art, such as high performance liquid chromatography (HPLC).

Specific probes for identifying such sequences as the WT1 encoding sequence may be between 15 and 40 nucleotides in length. For probes longer than those described above, the additional contiguous nucleotides are provided within the sequence encoding WT1.

In accordance with the present invention, nucleic acids having the appropriate level of sequence homology with the sequence encoding WT1 may be identified by using hybridization and washing conditions of appropriate stringency. For example, hybridizations may be performed, according to the method of Sambrook et al. (61), using a hybridization solution comprising: 5×SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

The nucleic acid molecules of the invention include cDNA, genomic DNA, RNA, and fragments thereof which may be single- or double-stranded. Thus, this invention provides oligonucleotides having sequences capable of hybridizing with at least one sequence of a nucleic acid molecule of the present invention, such as selected segments of the sequence encoding WT1. Also contemplated in the scope of the present invention are oligonucleotide probes which specifically hybridize with the DNA from the sequence encoding WT1 under high stringency conditions. Primers capable of specifically amplifying the sequence encoding WT1 are also contemplated to be within the scope of the present invention. As mentioned previously, such oligonucleotides are useful as primers for detecting, isolating and amplifying sequences encoding WT1.

WT1-encoding nucleic acids may be used for a variety of purposes in accordance with the present invention. WT1-encoding DNA, RNA, or fragments thereof may be used as probes to detect the presence of and/or expression of genes encoding WT1 proteins. Methods in which WT1-encoding nucleic acids may be utilized as probes for such assays include, but are not limited to: (1) in situ hybridization; (2) Southern hybridization (3) northern hybridization; and (4) assorted amplification reactions such as polymerase chain reactions (PCR). Detailed methodology for the above protocols are provided in a variety of basic laboratory manuals including: Sambrook et al (61) and Ausubel et al (62).

II. WT1 Antibodies

Polyclonal or monoclonal antibodies immunologically specific for WT1 protein may be used in a variety of assays designed to detect and quantitate the protein. Such assays include, but are not limited to: (1) flow cytometric analysis; (2) immunochemical detection/localization of WT1 protein in tumor cells or tissue samples; and (3) immunoblot analysis (e.g., dot blot, Western blot) of extracts from various cells. Such WT1 specific antibodies are commercially available (see Example I). Additionally, as described above, anti-WT1 antibodies can be used for purification of WT1 protein and any associated subunits (e.g., affinity column purification, immunoprecipitation). Detailed methodology for the protocols involving antibodies are provided in a variety of basic laboratory manuals including: Harlow and Lane (63).

III. Methods of Use for WT1 Antibodies and WT1 Primers and Kits for Performing the Disclosed Methods.

From the foregoing discussion, it can be seen that the invention provides methods wherein anti-WT1 antibodies can be used to detect WT1 protein levels and cellular localization, thereby providing means to diagnose RMS. Also provided are methods wherein WT1-encoding nucleic acids (i.e., primers) can be used to detect WT1 gene expression levels. The detection of WT1 gene expression levels provides means to determine the therapeutic efficacy of treatments administered to combat RMS, detect minimal residual disease or disease relapse, and anticipate the onset of RMS in relatives of patients diagnosed with RMS.

Exemplary approaches for detecting WT1 nucleic acid or polypeptides/proteins include:

a) determining the presence, in a sample from a patient, of the polypeptide encoded by the WT1 gene and, if present, determining whether the polypeptide is localized to the cytoplasm; and

b) using PCR involving one or more primers based on the WT1 gene sequence to screen for WT1 transcript levels in a sample from a patient.

Additional methods for PCR-mediated detection of WT1 transcript levels in peripheral blood and sera have been described by Menssen et al. (30) and Inoue et al. (21), the entire contents of which are incorporated herein by reference.

A “specific binding pair” comprises a specific binding member (sbm) and a binding partner (bp) that have a particular specificity for each other and which in normal conditions bind to each other in preference to other molecules. Examples of specific binding pairs are antigens and antibodies, ligands and receptors and complementary nucleotide sequences. The skilled person is aware of many other examples and they do not need to be listed here. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

The present inventor has discovered that high levels of circulating WT1 transcript are correlated with RMS. In one embodiment, the methods of the present invention, such as disclosed and discussed herein for establishing the presence or absence of WT1 transcript in a test sample, may be used to evaluate the therapeutic efficacy of a treatment for the eradication of RMS. The absence of WT1 transcripts in peripheral blood and sera may be used as a positive prognostic indicator for such patients. The presence of WT1 transcripts in peripheral blood and sera may be used as an indicator that the RMS cancer cells (e.g., a subpopulation) may be resistant to the therapeutic treatment, and/or of the presence of minimal residual disease or disease relapse. Indications of any of the above provide an attending physician with information critical in the treatment of a patient. Such information may be used to assess the potential for continued or different therapeutic intervention modalities. The method may also be used for diagnosing the predisposition of an individual for cancer. Evaluating the predisposition of an individual for cancer is particularly critical for relatives (e.g., siblings) of cancer patients. Siblings, in particular, have an increased risk for developing similar cancers and, thus, would benefit most from regular monitoring which provides early detection of disease onset.

In most embodiments for screening for RMS susceptibility in relatives of rhabdomyosarcoma patients or detecting WT1 transcript levels in samples derived from rhabdomyosarcoma patients, the WT1 nucleic acid in the sample will initially be amplified, e.g. using PCR, to increase the amount of the analyte as compared to other sequences present in the sample. This allows the target sequences to be detected with a high degree of sensitivity if they are present in the sample. This initial step may be avoided by using highly sensitive array techniques that are becoming increasingly important in the art.

In still further embodiments, the present invention concerns immunodetection methods for binding, purifying, removing, quantifying or otherwise generally detecting biological components. WT1 antibodies, for example, may be employed to detect high levels of WT1 protein localized in the cytoplasm of a biological sample.

In general, the immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing a WT1 gene encoded protein, peptide or a corresponding antibody, and contact the sample with an antibody or encoded protein or peptide, as the case may be, and then detect or quantify the amount of immune complexes formed under the specific conditions. In terms of antigen detection, the biological sample analyzed may be any sample that is suspected of containing the WT1 antigen, such as a tumor tissue section or specimen, a homogenized tumor tissue extract, an isolated cell, a cytosolic preparation, a cell membrane preparation, separated or purified forms of any of the above protein-containing compositions, or even any biological fluid that comes into contact with tumor tissues, including blood and lymphatic fluid.

Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The immunodetection methods of the present invention have evident utility in the diagnosis of RMS. Herein, a biological or clinical sample suspected of containing either the encoded protein or peptide or corresponding antibody is used.

In the clinical diagnosis of patients with RMS, the detection of high levels of WT1 antigen in the cytoplasm of a sample derived from a soft tissue tumor is indicative of a patient with RMS. The basis for such diagnostic methods lies, in part, with the finding of the present invention that elevated levels of WT1 protein staining localized to the cytoplasm of a cell is correlated with the presence of RMS in tissue samples. Moreover, the methods of the present invention relating to the detection of WT1 transcripts in peripheral blood and/or sera may also be used to detect minimal residual disease or disease which is innately resistant or has acquired resistance to a particular treatment. As such, the methods of the present invention provide means to monitor the efficacy of a therapeutic regime.

In one broad aspect, the present invention encompasses kits for use in detecting expression of WT1 in samples derived from soft tissue tumors. Such a kit may comprise antibodies or antibody fragments immunologically specific for WT1 proteins and means for assessing the formation of immunocomplexes containing WT1 protein in soft tissue tumor cells (i.e., RMS cells).

Another embodiment of the present invention encompasses a kit that includes one or more pairs of primers for amplifying nucleic acids corresponding to the WT1 gene. The kit may further comprise samples of total mRNA derived from tissue of various physiological states, such as normal and RMS samples, for example, to be used as controls. The kit may also comprise buffers, nucleotide bases, and other compositions to be used in hybridization and/or amplification reactions. Each solution or composition may be contained in a vial or bottle and all vials held in close confinement in a box for commercial sale.

The examples presented below are provided to illustrate certain embodiments of the invention. They are not intended to limit the invention in any way.

### EXAMPLE I

The following materials and methods are provided to facilitate the practice of the present invention.

Samples: Sixty-six formalin fixed and paraffin embedded tumor biopsy specimens were retrieved from the files at The Children's Hospital of Philadelphia. These specimens included eight peripheral neuroectodermal tumors (PNET/Ewing's), eight neuroblastomas, five desmoplastic small round blue cell tumors (DSRCTs), ten lymphomas, twenty-four Wilms' tumors and eleven rhabdomyosarcomas (RMS). The lymphomas included seven lymphoblastics, three Burkitt's, one large cell and one anaplastic. Five Wilms' tumors contained areas of heterologous differentiation (muscle or bone). The rhabdomyosarcoma cases included four embryonal, six alveolar and one para-testicular tumor. Supporting immunohistochemical stains (desmin, muscle specific actin, NSE, MIC2, O13, LCA, CD20, UCHL-1, CD30, EMA) and molecular translocation studies (PAX3/PAX7-FKHR, EWS-FLI1/ERG, EWS-WT1) were performed as needed in every case to confirm the diagnosis.

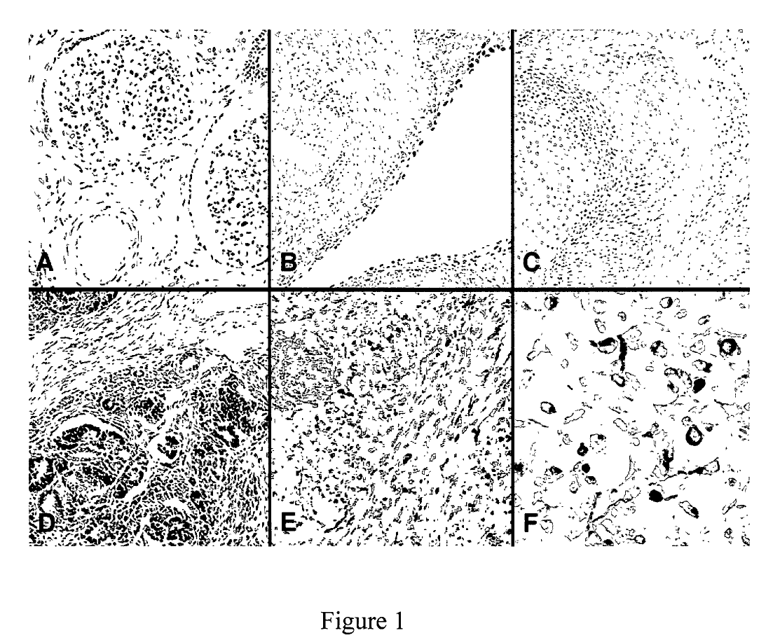

Immunohistochemistry: The WT1 antibody used was a mouse monoclonal (Dako Corporation: Carpinteria, Calif. 93013 USA; clone: 6F-H2) raised against the N-terminal 1-181 amino acids of human WT1 (43). The antibody was freshly diluted at 1:50 in PBS each time it was utilized. The antigen retrieval protocol required trypsin digestion for 20 minutes in a 37° C. oven. The antibody was detected by a standard avidin-biotin method. Normal kidney tissue (FIG. 1A) and an 8 weeks old human fetus (FIGS. 1B-1D) were used as controls. The results (Table 1) were graded as “0” if negative or as positive on a scale ranging from 1+ to 4+. This scale was based on the intensity (weak or elevated) and pattern (focal, multi-focal or diffuse) of staining. The Wilms' tumors received three grades. One for the epithelial component, one for the stromal and a third labeled as “highest score” (HS). The HS was based on the highest grade of the two components (Table 2).

Western blot analysis: 293T transformed kidney epithelial cells were transiently transfected with an expression construct encoding human WT-1, isoform B (pcDNA3-WT1B, kindly provided by Daniel Haber, Massachusetts General Hospital Cancer Center) using calcium phosphate precipitation. After 48-72 hours, cells were rinsed once with cold PBS, and harvested by scraping. Whole cell lysates were prepared from 293T cells, a Wilms' tumor and a rhabdomyosarcoma sample using lysis buffer (1% NP-40; 150 mM NaCl; 50 mM Tris, pH 7.4) containing protease inhibitors. Proteins were separated by SDS-PAGE and transferred to nitrocellulose. After blocking in 5% BSA-TBST for 1 hour at room temperature, Western blot analysis was performed using either anti-WT1 (clone 6F-H2, Dako, Carpinteria Calif.) or anti-tubulin (Santa Cruz Biotechnology, Santa Cruz Calif.) antibodies, according to standard protocols.

RT-PCR: Tumor tissue and peripheral blood was snap frozen in liquid nitrogen and stored at −70° C. until the time of testing. The tissue was homogenized and washed twice in PBS. Ten million cells were lysed with 1 ml Trizol reagent and RNA was isolated according to the manufacturer's protocol. One microgram of total RNA from each sample was reversed transcribed into cDNA according to standard protocols (Perkin Elmer Biosystem, CA). RT-PCR amplification of WT1 was performed using the following primers: 5′-GGCATCTGAGACCAGTGAG AA-3′ (SEQ ID NO: 1; outer sense), 5′ GAGAGTCAG ACTTGAAAGCAGT-3′ (SEQ ID NO: 2; outer antisense), 5′-GCT GTCCCACTTACAGATGCA-3′ (SEQ ID NO: 3; inner sense), 5′TCAAAGCGCCAGCTGGAGTTT-3′ (SEQ ID NO: 4; inner antisense). First round of PCR was carried out with 30 amplification cycles, followed by a second round of 30 cycles with a DNA thermal cycler (Perkin Elmer R480, CA). PCR products were separated in 1.5% of agarose gel. RNA obtained from either a K562 cell line or from a patient diagnosed with acute lymphoblastic leukemia (ALL) was used as a positive control. The ratio of WT1 and human glyceraldehyde 3-phosphate dehydrogenase (GAPDH) mRNA was used as an indication of the load and integrity of mRNA in the samples.

Results

The positive and negative controls stained as expected. The glomerular podocytes (FIG. 1A) and the mesothelial cells (FIG. 1B) nuclei were positive. The fetal skeletal muscle was negative (FIG. 1C).

The staining in the different SRBCT was variable in intensity (Table 1) and location (Table 2). A total of 66 cases were stained and 36% (24/66) were negative. The latter included eight (100%) PNET/Ewing's, five (71%) neuroblastomas, six (66%) lymphomas, two (40%) DSRCT, and two (8%) Wilms' tumors. The mildly positive cases (1+ or 2+) stained in a nuclear or cytoplasmic pattern and included two (25%) neuroblastomas, three (60%) DSRCT, and four (40%) lymphomas.

TABLE 1
Grading of WT1 (6F-H2) stain.

| GRADING | PATTERN |
|---|---|
| 0 | No staining |
| +1 | Weak and focal |
| +2 | Weak and multi-focal or diffuse |
| +3 | Elevated and focal or multifocal |
| +4 | Elevated and diffuse |

TABLE 2
WT1 (6H-F2) immunohistochemistry in small round blue cell tumors.

| | | | | | WT | | | |
|---|---|---|---|---|---|---|---|---|
| Tumor | PNET | NBL | DSRCT | LYMPH | 24 | | | RMS |
| (n = 66) | 8 | 8 | 5 | 10 | Ep-Bl | St | HS* | 11 |
| Negative | 8 | 6 | 2 | 6 | 10 | 7 | 2 | 0 |
| Nuclear | | | | | | | | |
| 1+ | | 1 | 1 | 1 | 4 | 1 | 2 | |
| 2+ | | | | 1 | 2 | | 2 | |
| 3+ | | | | | 3 | | 3 | |
| 4+ | | | | | 4 | | 4 | |
| Cytoplasmic | | | | | | | | |
| 1+ | | 1 | 2 | 1 | | 4 | 1 | |
| 2+ | | | | 1 | 1 | 9 | 6 | |
| 3+ | | | | | | 1 | 1 | 5 |
| 4+ | | | | | | 3 | 3 | 6 |

n: number of cases; PNET: Ewing's sarcoma; NBL: Neuroblastoma; DSRCT: desmoplastic small round blue cell tumor; LYMPH: Lymphoma; WT: Wilms' tumor; Ep: epithelial; Bl: blastema; St: stroma; HS: highest score (*see methods); RMS: rhabdomyosarcoma.

The Wilms' tumors had a variable nuclear (usually epithelial or blastemal) and/or cytoplasmic (usually stromal) positivity in 92% (22/24) of cases. The nuclear positivity (FIG. 1D) was recorded in 58% (14/24) and the cytoplasmic pattern was seen in 75% (18/24) of cases. Eleven (46%) Wilms' tumors had a weak positivity (1+ or 2+) based on the “highest score” criteria (see methods). Elevated nuclear positivity (+3 and +4) was confined to another eleven (46%) Wilms' tumors. In addition, all (5/5) Wilms tumors with muscle (heterologous) differentiation were positive in a cytoplasmic pattern (FIG. 1E) which correlated with areas of desmin reactivity.

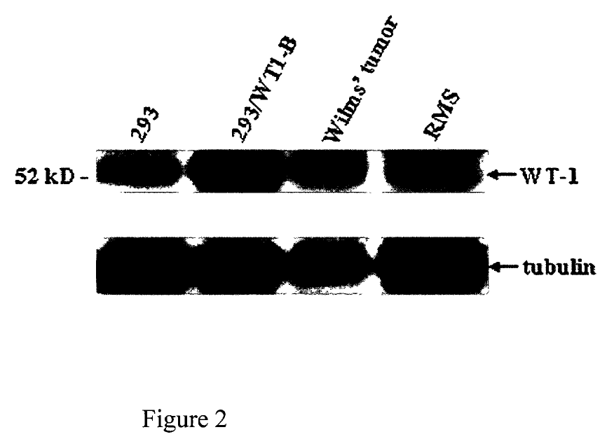

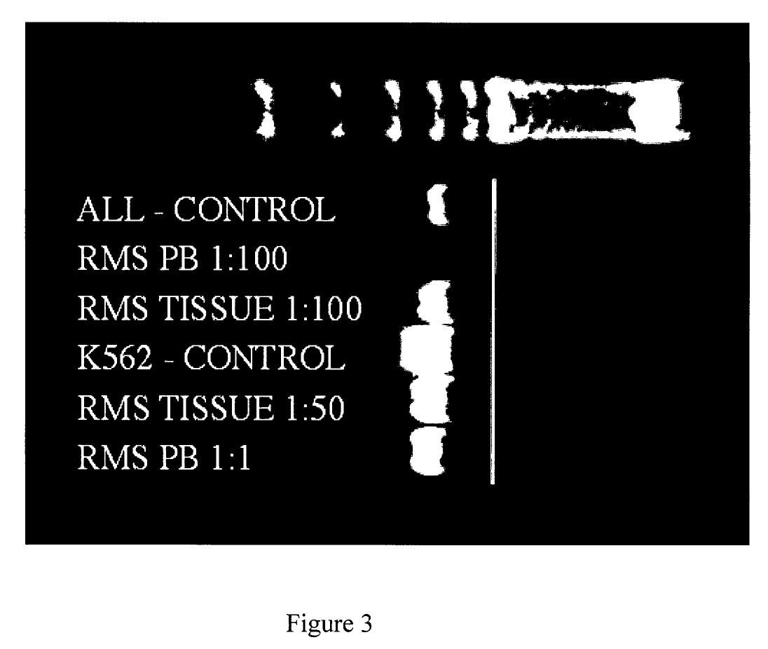

More interestingly, all (100%) rhabdomyosarcomas revealed an elevated cytoplasmic staining (FIG. 1F). The expression of WT1 was confirmed by Western blot analysis (FIG. 2). A band of 52 kD from the RMS cell lysate co-migrated with the endogenous WT-1 (293) in lysates from Wilms' tumor and an immunoreactive band in lysates from WT-1 (293/WT-1B) transfected cells. RT-PCR (FIG. 3) analysis of transcripts derived from RMS tissue (1:50 and 1:100 dilutions) and from undiluted peripheral blood (1:1) revealed a distinct and elevated band that co-migrated with that of known positive controls (K562 and ALL). The diluted peripheral blood (1:100 dilution) revealed a very weak band.

Discussion

Herein is provided the first report describing the diagnostic utility of a WT1 antibody (6H-F2), immunospecific for the N-terminal portion of WT1, in a large cohort of SRBCT. The most interesting aspect of this study was the elevated cytoplasmic staining in the rhabdomyosarcomas.

Previous immunohistochemical reports have described a weaker cytoplasmic positivity with WT1 antibodies in mesotheliomas (1, 24), leukemias (31) and in a few other small round blue cell tumors (4). The diagnostic utility of the 6H-F2 antibody was underscored by the consistently strong immunhistochemical pattern, and confirmed by the immunoblot results and RT-PCR analysis the tumor tissue. The absence of any reactivity with the normal fetal skeletal muscle and the expected specificity and pattern with mesothelial cells and podocytes provides further support for the immunohistochemical results.

Molecular studies have provided some insights into protein regulation that may be applicable to aberrant expression of WT1. Protein phosphorylation, for example, has been recognized as an important and, in some cases essential, regulatory component for nuclear translocation or cytoplasmic sequestration of transcription factors. Indeed, cytoplasmic expression WT1 has recently been demonstrated by “in vitro” phosphorylation of transfected cell lines (56). In this study, as documented by immunofluorescence with a C19 (C-terminal) WT1 antibody, protein kinase A (PKA)-catalyzed phosphorylation induced by forskolin resulted in the appearance of WT1 cytosolic staining in 197/200 cells. Another study (28) suggested that WT1 could be sequestered along with p53 as a “cytoplasmic body” in adenovirus-transformed kidney cells. The degree of expression noted in tumors of the present study, however, was quite disparate from that described in any previous reports.

Rhabdomyogenesis is common in Wilms' tumors and correlates with a younger age and more favorable outcome. In contrast, many previous reports (6, 9, 11, 15, 16, 29, 49, 54) noted a possible linkage between Wilms' tumors and rhabdomyosarcomas. A recent investigation (32) using cell lines derived from a panel of Wilms' tumors attempted to analyze the role of WT1 in the myogenic program. The authors of this study demonstrated that complete loss of WT1 could lead to muscle differentiation in a few cases. In contrast, a larger cohort of tumors lacking WT1 gene mutations rarely showed MyoD1, myosin heavy polypeptide 3 (MYH3) or myogenin (MYOG) expression. These findings, in conjunction with in situ studies, suggested that WT1 expression in the metanephric-mesenchymal stem cells of chick and mice kidneys probably inhibited the skeletal muscle differentiation of these cells. Additional studies have correlated WT1 mutations (41, 47) with stromal predominant Wilms' tumors. One report (12) noted a relatively lower level of WT1 RNA transcripts in tumors with heterologous elements.

The findings presented herein suggest that WT1 properties may be deregulated in Wilms' tumor cells. WT1 in Wilms' tumors may not exhibit normal activity because it is no longer competent to bind nuclear DNA as a result of mutations in the zinc finger region. Observations presented herein further suggest that the protein is functioning abnormally in rhabdomyosarcomas by virtue of its stabilization in and localization to the cytoplasm.

In summary, this study reveals that when used in immunohistochemistry studies of paraffin embedded tissues, the 6F-H2 antibody is a useful adjunct in the differentiation of Wilms' tumors and rhabdomyosarcomas from other small round blue cell tumors. The elevated cytoplasmic expression correlates with muscle differentiation and supports the idea that WT1 is deregulated in rhabdomyosarcomas. Future studies may explain the discrepant staining pattern between N-terminal and C-terminal specific WT1 antibodies. WT1 sequencing, phosphorylation studies, peripheral blood testing (RNA and/or serology), and immunotherapy may be of value in patients diagnosed with rhabdomyosarcoma.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

References:

1. Amin K M, Litzky L A, Smythe W R, et al. Wilms' Tumor 1 susceptibility (WT1) gene products are selectively expressed in malignant mesothelioma. Am J Pathol 1995;146(2):344-356.

2. Armstrong J F, Pritchard-Jones K, Bickmore W, Hastie N D, Bard J B L. The expression of the Wilms' tumor gene, WT1, in the developing mammalian embryo. Mech Dev 1992;40:85-97.

3. Baird P N, Simmons P J. Expression of the Wilms' tumor gene (WT1) in normal hemopoiesis. Exp Hematol 1997;25:312-320.

4. Barnoud R, Sabourin J- C, Pasquier D, Ranchere D, Bailly C, Terrier-Lacombe M- J, Pasquier B. Immunohistochemical expression of WT1 by desmoplastic small round cell tumor. A comparative study with other small round cell tumors. Am J Surg Pathol 2000; 24(6);830-36.

5. Bayani J, Thorner P, Zielenska M, Pandita A, Beatty B, Suire J A. Application of a simplified comparative genomic hybridization technique to screen for gene amplification in pediatric solid tumors. Ped Pathol Lab Med 1995;15(6):831-44.

6. Besnard-Guerin C, Newsham I, Winqvist R, Cavenes W K. A common region of loss of heterozygosity in Wilms' tumor and embryonal rhabdomyosarcoma distal to the D11S988 locus on chromosome 11p15.5 Hum Genet February 1996;97(20:163-170.0

7. Denamur E, Bocquet N, Mougenot B, et al. Mother-to-child transmitted WT1 splice-site mutation is responsible for distinct glomerular diseases. J Am Soc Nephrol 1999;10(10): 2219-23.

8. Englert C. WT1-more than a transcriptional factor? TIBS 1998;23:389-393.

9. Folpe A L, Patterson K, Gown A M. Antibodies to desmin identify the blastemal component of nephroblastoma. Mod Pathol September 1997;10(9):895-900.

10. Gaiger A, Carter L, Greinix H, et al. WT1-specific serum antibodies in patients with leukemia. Clin Cancer Res March 2001;7(3 Suppl):761s-765s.

11. Garvin A J, Surrete F, Hintz D S, Rudisill M T, Sens M A, Sens D A. The in vitro growth and characterization of the skeletal muscle component of Wilms' tumor. Am J Pathol November 1985;121(2):298-310.

12. Gerald W L, Gramling S, Sens D A, Garvin J. Expression of the 11p13 Wilms' tumor gene, WT1, correlates with histologic category of Wilms' tumor. Am J Pathol 1992;140(5):1031-1037.

13. Gessler M, Konig A, Bruns G A P. The genomic organization and expression of the WT1 gene. Genomics 1992;12:807-813.

14. Goldstein N S, Bassi D, Uzieblo A. WT1 is an integral component of an antibody panel to distinguish pancreatobiliary and some ovarian epithelial neoplasms. Am J Clin Pathol August 2001;116(2):246-52.

15. Gonzales-Crussi F, Hsueh W, Ugarte N. Rhabdomyogenesis in renal neoplasia of childhood. Am J Surg Pathol September 1981;5(6):525-32.

16. Gough D C. Wilms' tumour and genitourinary rhabdomyosarcoma. Br J Urol February 1989;63(2):109-16.

17. Haber D A, Englert C, Maheswaran S. Functional properties of WT1. Med Ped Oncol 1996;27:453-455.

18. Hastie N D. The genetics of Wilms' tumor-A case of disrupted development. Annu Rev Genet. 1994;28:523-58.

19. Hill D A, Pfeifer J D, Marley E F, et al. WT1 staining reliably differentiates desmoplastic small round blue cell tumor. An immunohistochemical and molecular diagnostic study. Am J Clin Pathol. 2000;114:345-353.

20. Im H J, Kong G, Lee H. Expression of Wilms' tumor Gene (WT1) in children with acute leukemia. Ped Hematol Oncol 1999;16:109-118.

21. Inoue K, Ogawa H, Sonoda Y, et al. Aberrant expresion of the Wilms' tumor gene (WT1) in human leukemia. Blood 1997;89(4):1405-1412.

22. Klamt B, Koziell A, Poulat F, Wieacker P, Scambler P, Berta P, Gessler M. Frasier syndrome is caused by defective alternative splicing of Wt1 leading to an altered ratio of Wt1 +/−KTS splice isoforms. Hum Molec Genet 1998;7(4):709-14.

23. Kreidberg J A. Gene targeting in kidney development. Med Ped Oncol 1996;27:445-452.

24. Kumar-Singh S, Segers K, Rodeck U, et al. WT1 mutation in malignant mesothelioma and WT1 immunoreactivity in relation to p53 and growth factor receptor expression, cell-type transition, and prognosis. J Pathol 1997;181:67-74.

25. Loeb D M, Evron E, Patel C B, et al. Wilms' tumor suppressor gene (WT1) is expressed in primary breast tumors despite tumor-specific promoter methylation. Cancer Res Feb. 1, 2001;61(3): 921-5.

26. Loke S L, Neckers L M, Schwab G, Jaffe E. c-myc protein in normal tissue. Effects of fixation on its apparent subcellular distribution. Am J Pathol 1988;131:29-37.

27. Machin G A. Atypical presentation of Denys-Drash syndrome in a female with a novel WT1 gene mutation. Birth Defects 1996;30(1):269-286.

28. Maheswaran S, Englert C, Lee S B, Ezzel R M, Settleman J, Haber D A. E1B 55K Sequesters WT1 along with p53 within a cytoplasmic body in adenovirus-transformed kidney cells. Oncogene 1998;16(16):2041-50.

29. Marcus R, Perez-Atayde A R. Unique dermal and subcutaneous botryoid rhabdomyosarcoma associated with mature renal tissue: is this an extrarenal Wilms' tumor? Pediatr Pathol July-August 1994;14(4):617-25.

30. Menssen H D, Renkl H J, Rodeck U, et al. Presence of Wilms' tumor gene (wt1) transcripts and the WT1 nuclear protein in the majority of human acute leukemias. Leukemia 1995;9:1060-1067.

31. Menssen H D, Renkl H- J, Rodeck U, Kari C, Schwartz S, Thiel E. Detection by monoclonal antibodies of the Wilms' tumor (WT1) nuclear protein in patients with acute leukemia. Int J Cancer 1997;70:518-523.

32. Miyagawa K, Kent J, Moore A, et al. Loss of WT1 function leads to ectopic myogenesis in Wilms' tumour. Nat Genet 1998;18:15-17.

33. Miyagawa K, Hayashi Y, Fukuda T, Mitani K, Hirai H, Kamiya K. Mutations of the WT1 gene in childhood nonlymphoid hematological malignancies. Genes Chrom Cancer 199;25(2):176-183.

34. Moffett P, Bruening W, Nakagama H, et al. Antagonism of WT1 activity by protein self-association. Proc Natl Acad Sci USA 1995;92(24:11105-9.

35. Moore Aw, Schedl A, McInnes L, Doyle M, Hecksher-Sorensen J, Hastie N D. YAC transgenic analysis reveals Wilms' tumour 1 gene activity in the proliferating coelomic epithelium, developing diaphragm and limb. Mech Dev December 1998;79(1-2):169-84.

36. Niegemann E, Wehner S, Kornhuber B, Schwabe D, Ebener U. wt1 Gene expression in childhood leukemias. Acta Haematol 1999; 102(2):72-6.

37. Park S, Schalling M, Bernard A, et al. The Wilms' tumour gene WT1 is expressed in murine mesoderm-derived tissues and mutated in a human mesothelioma. Nat Genet 1993;4:415-20.

38. Pelletier J, Bruening W, Li F P, Haber D A, Glaser T, Housman D E. WT1 mutations contribute to abnormal genital system development and hereditary Wilms' tumour. Nature 1991;353(6343):431-4.

39. Peter M, Gilbert E, Delattre O. A multiplex real-time pcr assay detection of gene fusions observed in solid tumors. Lab Invet June 2001;81(6):905-12.

40. Poulat F, Morin D, Konig A, et al. Distinct molecular origins for Denys-Drash and Frasier syndromes. Hum Genet 1993;91(3):285-6.

41. Pritchard-Jones K, Fleming S. Cell types expressing the Wilms' tumour gene (WT1) in Wilms' tumours: Implications for tumour histogenesis. Oncogene 1991;6:2211-2220.

42. Ramani P. Cowell J K. The expression pattern of Wilms' tumour gene (WT1) product in normal tissues and paediatric renal tumours. J Pathol June 1996,179(2):162-8.

43. Rauscher III, F J, Morris J, Fredericks W J, et al. Characterization of monoclonal antibodies directed to the amino-terminus of the WT1, Wilms' tumor supressor protein. Hybridoma 1998;17(2):191-198.

44. Reddy J C, Morris J C, Wang J, English M A, Haber D A, Shi Y, Licht J D. WT1 mediated transcriptional activation is inhibited by dominant negative mutant proteins. J Biol Chem 1995;270(18):10878-84.

45. Satoh F, Tsutsumi Y, Yokoyama S, et al. Comparative immunohistochemical analysis of developing kidneys, nephroblastomas and related tumors: considerations on their pathogenesis. Pathol Int June 2000;50(6):458-71.

46. Scharnhorst V, Dekker P, van der Eb A J, Jochemsen A G. Internal translation initiation generates novel WT1 protein isoforms with distinct biological properties. J Biol Chem 1999;274(33):23456-62.

47. Schumacker V, Schneider S, Figge, et al. Correlation of germ-line mutations and Two-Hit inactivation of the WT1 gene with Wilms tumors of stromal-Predominant Histology. Proc Natl Acad Sci USA 1997;94:3972-3977.

48. Silberstein G B, Van Horn K, Strickland P, Roberts Jr, C T, Daniel C W. Altered expression of the WT1 Wilms tumor supressor gene in human breast cancer. Proc Natl Acad Sci USA 1997;94:8132-8137.

49. Steenman M, Tomlinson G, Westerveld A, Mannens M. Comparative genomic hybridization analysis of hepatoblastomas: additional evidence for a genetic link with Wilms tumor and rhabdomyosarcoma. Cytogenet Cell Genet 1999;86(2);157-161.

50. Sugiyama H. Wilms' tumor gene WT1: its oncogenic function and clinical application. Int J Hematol February 2001;73(2):177-87.

51. Thorner P, Squire J, Plavsic N, Jong R, Greenberg M, Zielenska M. Expression of WT1 in pediatric small cell tumors: Report of two cases with a possible mesothelial origin. Ped Dev Pathol 1999;291:33-41.

52. Thorner P S, Squire J A. Molecular genetics in the diagnosis and prognosis of solid pediatric tumors. Persp Ped Pathol 1999;21:70-98.

53. Tsuboi A, Oka Y, Ogawa H, et al. Cytotoxic T-lymphocyte responses elicited to Wilms' tumor gene WT1 product by DNA vaccination. J Clin Immunol May 2000;20(3): 195-202.

54. Vawter G F. Anaplastic Wilms' tumor with rhabdomyosarcomatous differentiation. Pediatr Pathol January-February 1991;11(1);177-82.

55. Yang Y, Jeanpierre C, Dressler G R, Lacoste M, Niaudet P, Gubler M C. WT1 and PAX-2 podocyte expression in Denys-Drash syndrome and isolated diffuse mesangial sclerosis. Am J Pathol 1999;154:181-192.

56. Ye Ying, Raychaudhuri B, Gurney A, Campbell C E, Williams B R G. Regulation of WT1 by Phosphorylation: Inhibition of DNA binding, Alteration of transcriptional activity and cellular translocation. EMBO 1996; 15(20):5606-5615.

57. Chen Z, Coffin C M, Smith L M, Issa B, Arndt S, Shepard R, Brothman L, Stratton J, Brothman A R, Zhou H. Cytogenetic-clinicopathologic correlations in rhabdomyosarcoma. A report of five cases. Cancer Genet Cytogenet 2001; 131:31-36.

58. Armstrong S J, Duncan A W, Mott M G. Rhabdomyosarcoma associated with familial adenomatous polyposis. Pediatr Radiol 1991; 21:445-446;

59. Diller L. Rhabdomyosarcoma and other soft tissue sarcoma of childhood. Curr Opin Oncol 1992; 4:689-695;

60. Lustbader E D, Williams W R, Bondy M L, Strom S, Strong L C. Segregation of cancer in families of childhood soft-tissue-sarcoma patients. Am J Hum Genet 1992; 51:344-356

61. Sambrook J, Fritsch E F, Maniatis T. (eds.) Molecular Cloning A Laboratory Manual. Second Edition. 1989. Cold Spring Harbor Laboratory Press.

62. Ausubel F M, Brent R, Kingston R E, Moore D D, Seidman J G, Smith J A, Struhl K. (eds.) Current Protocols in Molecular Biology, 1992. J W Wiley and Sons, NY.

63. Harlow E and Lane D. Antibodies A Laboratory Manual, 1988. Cold Spring Harbor Laboratory Press.

What is claimed is:

1. A method for diagnosing pediatric soft tissue tumors based on differential localization of a WT1-associated molecule, said method comprising:
a) obtaining a biological sample from a pediatric patient;
b) contacting said sample with an agent having affinity for said WT1-associated molecule and;
c) determining a cellular localization of said WT1-associated molecule as indicated by localization of said agent having affinity for said WT1-associated molecule, wherein detection of elevated cytoplasmic levels of said WT1-associated molecule provides a positive diagnostic indicator of a rhabdomyosarcoma tumor.

2. The method as claimed in claim 1, wherein said WT1-associated molecule is selected from the group consisting of a WT1 polypeptide, a WT1 nucleic acid or fragments thereof.

3. The method as claimed in claim 1, wherein said method further comprises a method selected from the group consisting of a method for detecting myogenin, MyoD, desmin, muscle-specific actin, and myoglobin.

4. The method as claimed in claim 1, wherein said agent having affinity for a WT1-associated molecule comprises a detectable label.

5. The method as claimed in claim 4, wherein said detectable label is selected from the group consisting of fluorescein, rhodamine, phycoerythrin, biotin, and strepavidin.

6. The method as claimed in claim 1, wherein said agent having affinity for a WT1-associated molecule is detected by a method selected from the group consisting of flow cytometric analysis, immunochemical detection and immunoblot analysis.

7. The method of claim 1, wherein said agent having affinity for a WT1-associated molecule is in solution.

8. The method as claimed in claim 1, wherein said biological sample is selected from the group consisting of soft tissue tumor cells, rhabdomyosarcoma cancer cells, Wilms' tumor and non-malignant cells.

9. A method for diagnosing pediatric soft tissue tumors based on differential localization of WT1 protein, said method comprising:
a) obtaining a sample from a pediatric patient;
b) contacting said sample with an antibody or antibody fragment immunologically specific for WT1 protein; and
c) determining cellular localization of WT1 as indicated by localization of said antibody or antibody fragment immunologically specific for WT1, wherein elevated cytoplasmic WT1 staining provides a positive diagnostic indicator of a rhabdomyosarcoma tumor.

10. The method as claimed in claim 9, wherein said antibody is immunologically specific for an amino terminal region of WT1.

11. The method as claimed in claim 9, wherein said antibody comprises a detectable label.

12. The method as claimed in claim 11, wherein said detectable label is selected from the group consisting of fluorescein, rhodamine, phycoerythrin, biotin, and strepavidin.

13. The method as claimed in claim 9, wherein said antibody is detected by a method selected from the group consisting of flow cytometric analysis, immunochemical detection and immunoblot analysis.

14. The method of claim 9, wherein said antibody or fragment is in solution.

15. The method as claimed in claim 9, wherein said biological sample comprises soft tissue tumor cells and non-malignant cells.

16. The method of claim 9, wherein said biological sample comprises rhabdomyosarcoma cancer cells.

17. The method of claim 9, wherein said biological sample comprises Wilms' tumor cells.

18. A method for detecting WT1 encoding nucleic acid in a biological sample as a tumor marker for rhabdomyosarcoma cancer, wherein said biological sample is derived from a patient diagnosed with rhabdomyosarcoma, said method comprising:
a) extracting nucleic acids from said biological sample;
b) contacting said extracted nucleic acid with oligonucleotide primers which specifically hybridize to WT1 encoding nucleic acids if any are present;
c) subjecting said extracted nucleic acid and primers to conditions suitable for polymerase chain reaction amplification; and
d) assessing the resulting reaction product for amplified WT1 nucleic acid.

19. The method as claimed in claim 18, wherein said reaction product is assessed by a method selected from the group consisting of gel electrophoresis, restriction digest mapping, scintillation counting and filter paper assays.

20. The method as claimed in claim 18, wherein said primers have a sequence selected from the group consisting of: SEQ ID NO: 1, SEQ ID NO: 2, SEQ ID NO: 3, and SEQ ID NO: 4.

21. The method as claimed in claim 20, wherein said primers comprise a detectable label.

22. The method as claimed in claim 21, wherein said detectable label is selected from the group consisting of chemiluminescent, enzymatic, radioactive, fluorescent, biotin, and streptavidin.

23. The method as claimed in claim 18, wherein said biological sample is selected from the group consisting of rhabdomyosarcoma cancer cells, lymphatic cells, tumor cells, non-malignant cells, peripheral blood, and sera.

24. The method of claim 18, wherein said biological sample comprises peripheral blood.

25. The method of claim 18, wherein said biological sample comprises sera.

26. The method of claim 18, wherein said patient diagnosed with rhabdomyosarcoma is undergoing treatment for rhabdomyosarcoma.

27. The method of claim 18, wherein said patient diagnosed with rhabdomyosarcoma has completed treatment for rhabdomyosarcoma.

28. The method of claim 18, wherein said patient diagnosed with rhabdomyosarcoma is in remission.

29. The method as claimed in claim 18, wherein said method for detecting WT1 encoding nucleic acid in a biological sample as a tumor marker for rhabdomyosarcoma cancer comprises a method to monitor residual disease.

30. A kit for detecting WT1 in a biological sample, said kit comprising:
a) an antibody or fragment thereof immunologically specific for a region of WT1;
b) a detectable label for said antibody; and
c) reagents suitable for detecting WT1-antibody immunocomplexes, if present in said biological sample.

31. The kit as claimed in claim 30, wherein said antibody or fragment thereof is in solution.

32. The kit as claimed in claim 30, wherein said detectable label is selected from the group consisting of fluorescein, rhodamine, phycoerythrin, biotin, and strepavidin.

33. The kit as claimed in claim 30, optionally comprising reagents suitable for flow cytometric analysis, immunochemical detection and immunoblot analysis.

34. The method as claimed in claim 30, wherein said antibody is immunologically specific for an amino terminal region of WT1.

35. A kit for identifying WT1 in a biological sample, said kit comprising:
a) at least one pair of primers, said primers having the sequence selected from the group consisting of: SEQ ID NO: 1, SEQ ID NO: 2, SEQ ID NO: 3, and SEQ ID NO: 4;
b) a polymerase enzyme suitable for use in polymerase chain reaction;
c) buffers and nucleotides suitable for performing amplification reactions;
d) a DNA sample comprising a positive control; and
e) optionally an instruction protocol.

36. The kit as claimed in claim 35, wherein said primer comprises a detectable label.

37. The kit as claimed in claim 36, wherein said detectable label is selected from the group consisting of: chemiluminescent, enzymatic, radioactive, fluorescent, biotin, and streptavidin.

38. The kit as claimed in claim 35, optionally comprising reagents suitable for gel electrophoresis, restriction digest mapping, scintillation counting and filter paper assays.

39. The kit as claimed in claim 35, further comprising:
f) an antibody or fragment thereof immunologically specific for a region of WT1;
g) a detectable label for said antibody; and
h) reagents suitable for detecting WT1-antibody immunocomplexes, if present in said biological sample.
